(19) United States
(12) Patent Application Publication
(10) Pub. No.: US 2016/0325227 A1
(43) Pub. Date: Nov. 10, 2016

(54) Manufacturing Method of Urea Solution Used For Intelligent And High Efficiency Diesel Engine Vehicle

(71) Applicant: Vovax Material and Technology, Taoyuan (TW)

(72) Inventor: Lien-Chih Yang, Taoyuan (TW)

(21) Appl. No.: 14/704,158

(22) Filed: May 5, 2015

(51) Int. Cl. B01D 53/94; C07C 273/14

(52) U.S. Cl. CPC B01D 53/9409; C07C 273/14; B01D 2251/2067

(57) ABSTRACT

A manufacturing method of urea solution used for intelligent and high efficiency diesel engine vehicle, comprising following steps: utilizing a reverse osmosis (RO) water purification device to purify faucet water into purified water; utilizing a pre-heating device to pre-heat the purified water to 50° C.˜60° C.; adding urea material into pre-heated water, and using a stirring device to stir them, to make urea material dissolve into the pre-heated water, to form finished product of pure urea solution used for diesel engine vehicle. In this approach, through putting urea material into the pre-heated water to stir and mix them well, effect of urea material heat absorption is reduced, to raise urea solubility efficiency 5 times. Also, production space is reduced, such that production space can be moved around swiftly, in achieving reducing production cost, fast response to market demand, and raising production profit.

### BACKGROUND OF THE INVENTION

1. Field of the Invention

The present invention relates to a manufacturing method of urea solution used for intelligent and high efficiency diesel engine vehicle, and in particular to a urea solution manufacturing method, such that in the urea solution preparing process, the effect of its heat absorption can be reduced, to raise its solubility efficiency, while reducing the production space required, so that the production space can be moved around swiftly to realize Distributed Production. As such, the urea solution manufacturing method is capable of reducing production cost, achieving fast response to market demand, providing efficient service to the customers, and raising profit of production.

2. The Prior Arts

In general, in treating the exhaust expelled from the diesel engine vehicle, in order to meet the environment protection requirement, a Selective Catalystic Reduction (SCR) system is installed in a generator of a diesel engine, and a pure urea solution using as catalyst reduction agent is added in that SCR system, such that for a diesel engine having installed such a SCR system, the nitrogen oxide and particles contained in the exhaust of the diesel engine can be reduced, to meet the environment protection regulations of Euro 5, Euro 6, and US 2014 National Environment Production Standard.

The Specifications for the pure urea solution (AdBlue) utilized as catalyst reduction agent can be summarized in the following table 1:

TABLE 1
specifications for pure urea solution (AdBlue)
utilized as catalyst reduction agent

| Characteristics | Unit | Limits | Test methods |
|---|---|---|---|
| Urea content | %(m/m) | 31.8~33.2 | ISO 22241-2 Annex B |
| | | | ISO 22241-2 Annex C |
| Density at 20° C. | Kg/m³ | 1.087~1.093 | ISO 3675 or ISO 12185 |
| Refractive index | — | 1.3814~1.3843 | ISO 22241-2 Annex C |
| at 20° C. ° C. | | | |
| Alkalinity as NH₃ | %(m/m) | <0.2 | ISO 22241-2 Annex D |
| Biuret | %(m/m) | <0.3 | ISO 22241-2 Annex E |
| Aldehydes | mg/kg | <5.0 | ISO 22241-2 Annex F |
| Insoluble matter | mg/kg | >100 | ISO 22241-2 Annex G |
| Phosphate(PO₄) | mg/kg | >10 | ISO 22241-2 Annex H |
| Calcium | mg/kg | >2 | ISO 22241-2 Annex I |
| Iron | mg/kg | >2 | |
| Copper | mg/kg | >2 | |
| Zinc | mg/kg | >2 | |
| Chromium | mg/kg | >2 | |
| Nickel | mg/kg | >2 | |
| Aluminum | mg/kg | >2 | |
| Magnesium | mg/kg | >2 | |
| Sodium | mg/kg | >2 | |
| Potassium | mg/kg | >2 | |

In the Prior Art, in order to obtain pure urea solution used for diesel engine vehicle, a manufacturing method is used for this purpose, including the following steps: firstly, obtaining an industrial grade urea solution. Next, utilizing a front-stage filter system to preliminarily filter out the particles and impurities from the industrial grade urea solution. Then, utilizing resin contained in an ion exchange machine of the purification equipment to filter out further the particles and impurities in the industrial grade urea solution by absorbing them onto the resin. And finally, utilizing a rear-stage filter system to filter out once again the remaining particles and impurities, to obtain a purr urea solution used for a diesel engine vehicle.

Though in the Prior Art, the diesel engine vehicle urea solution manufacturing method is able to produce the pure urea solution for vehicle, yet its production requires to use equipment occupying large space, such that usually the manufacturing factory is located in a remote Industrial Zone. Therefore, the urea solution delivery cost is high, and it can not achieve prompt delivery to the customer due to long distance. In addition, in production, the solution of urea material in water requires long period of time, while the cost is high for treating high concentration waste water produced in the ion exchange process. Further, a large scale waste water treatment system is required to handle the problem of water pollution and environment protection, thus increasing production cost significantly, and not being able to achieve prompt delivery at low cost.

Therefore, presently, the design and performance of urea solution manufacturing method is not quite satisfactory, and it has much room for improvement.

### SUMMARY OF THE INVENTION

In view of the problems and drawbacks of the prior art, the present invention provides a manufacturing method of urea solution used for intelligent and high efficiency diesel engine vehicle, to overcome the shortcomings of the Prior Art.

The major objective of the present invention is to provide a manufacturing method of urea solution used for intelligent and high efficiency diesel engine vehicle, such that in the urea preparing process, the effect of its heat absorption can be reduced to raise its solubility efficiency, while reducing the production space required, so that it has the advantage of conducting production in the production space moving at high speed (for example, in a container on a container truck), and it is able to cover several service areas around. As such, the urea solution manufacturing method is capable of reducing production cost, achieving fast response to market demand, providing fast delivery to the customers, to win over the customers and serve them well, in achieving raised sales and production profit.

In order to achieve the objective mentioned above, the present invention provides a manufacturing method of urea solution used for intelligent and high efficiency diesel engine vehicle, including the following steps: firstly, utilizing a reverse osmosis (RO) water purification device to purify faucet water into purified water. Next, utilizing a pre-heating device to pre-heat the purified water to 50° C.˜60° C. Then, adding urea material of granular shape into the purified and pre-heated water, and using a stirring device to stir them to make the urea material dissolve into the pre-heated water, to form the finished product of urea solution used for diesel engine vehicle. Finally, storing the finished product of urea solution into a tank. In this way, through putting the urea material into the purified and pre-heated water and to stir and mix them well, the urea solubility efficiency can be raised more than 5 times, to shorten its solubility time required to about 20 minutes. Since, in the process of preparing the urea solution, the effect of its heat absorption is reduced, thus raising its solubility efficiency. In addition, the space required for production is decreased, such that the production space can be moved around swiftly (for example, in a container on a container truck). As such, the urea solution manufacturing method is capable of reducing production cost, achieving fast response to market demand, providing fast delivery to the customers, and raising profit of production.

Further scope of the applicability of the present invention will become apparent from the detailed descriptions given hereinafter. However, it should be understood that the detailed descriptions and specific examples, while indicating preferred embodiments of the present invention, are given by way of illustration only, since various changes and modifications within the spirit and scope of the present invention will become apparent to those skilled in the art from this detailed descriptions.

### BRIEF DESCRIPTION OF THE DRAWINGS

The related drawings in connection with the detailed descriptions of the present invention to be made later are described briefly as follows, in which:

FIG. 1 is a flowchart of the steps of a manufacturing method of urea solution used for intelligent and high efficiency diesel engine vehicle according to an embodiment of the present invention; and

FIG. 2 is schematic diagram of comparison for Mess Production and Distributed Production according to an embodiment of the present invention.

### DETAILED DESCRIPTION OF THE PREFERRED EMBODIMENT

The purpose, construction, features, functions and advantages of the present invention can be appreciated and understood more thoroughly through the following detailed description with reference to the attached drawings.

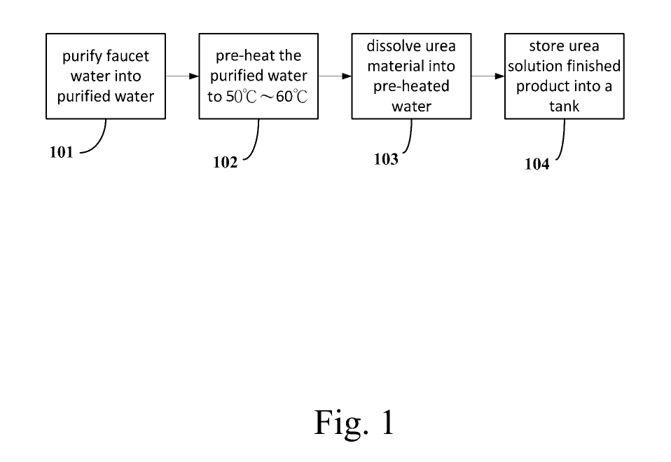

Refer to FIG. 1 for a flowchart of the steps of a manufacturing method of urea solution used for intelligent and high efficiency diesel engine vehicle according to an embodiment of the present invention. As shown in FIG. 1, the manufacturing method of urea solution includes the following steps: firstly, utilizing a reverse osmosis (RO) water purification device to purify faucet water into purified water (step 101). Next, utilizing a pre-heating device to pre-heat the purified water to about 50° C.˜60° C., while keeping that temperature to continue preheating the purified water (step 102). Then, adding high quality urea material of granular shape (containing biuret) and urea material containing low metal content into the pre-heated water, and using a stirring device to stir them, to make the urea material dissolve into the pre-heated water, to form the finished product of pure urea solution used for diesel engine vehicle (step 103). Finally, storing the finished product of pure urea solution into a tank (step 104).

In this approach, through putting the urea material into the purified and pre-heated water, and using a stirring device to stir and mix them well, the urea material solubility efficiency can be raised more than 5 times, while reducing the solubility time required to about 20 minutes. Since, in the process of preparing the urea solution, the effect of its heat absorption is reduced, thus raising its solubility efficiency and reducing production cost significantly.

Refer again to FIG. 1, the finished product of urea solution used for diesel engine vehicle thus produced can be put through a filter, to filter out the particles and impurities further, and then store it into a tank. Moreover, the pre-heating device can be a heat pump heater.

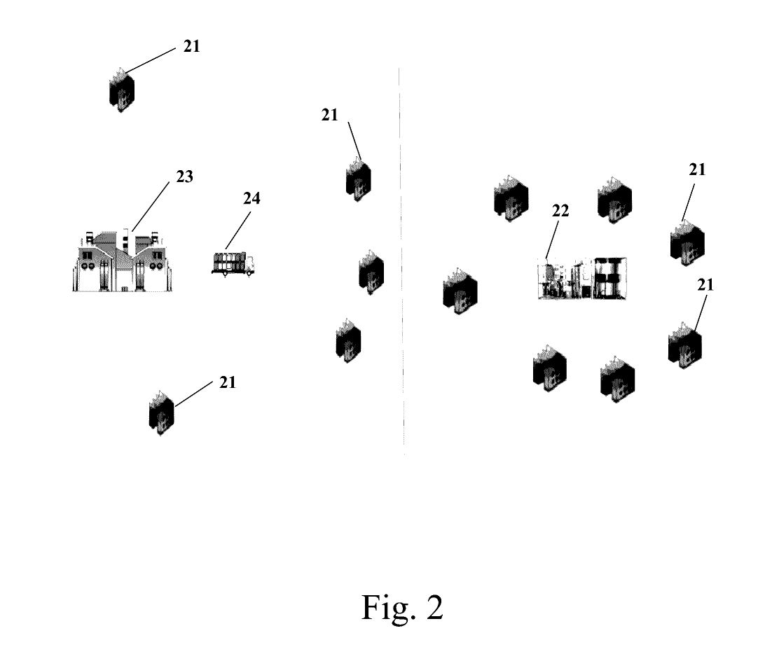

Then, refer to FIG. 2 for a schematic diagram of comparison for Mess Production and Distributed Production according to an embodiment of the present invention. As shown in FIGS. 1 and 2, the manufacturing method of urea solution can be used to transform the production of urea solution from Mess Production to Distributed Production. For the Distributed Production, each urea solution production station 22 can be arranged in a specific service area covering a plurality of customers 21 in that area, so that each urea solution production station 22 is able to deliver the urea solution to each of the customers 21 in a short distance in that service area, and to provide them with prompt service. Also, since the space used for production is reduced, so that it has the advantage of conducting production in the production space moving at high speed (for example, in a container on a container truck), and it is able to cover several areas around. As such, the urea solution manufacturing method is capable of reducing production cost, achieving fast response to market demand, providing fast delivery to the customers, to win over the customers and serve them well, in achieving raised sales and production profit.

Further, as shown in FIG. 2, on the left side is shown the schematic diagram of Mess Production, wherein, the manufacturing factory 23 is used to produce urea solution, while transport vehicle 24 is used to deliver the urea solution to the customers 21 around. On the right side of FIG. 2 is shown the schematic diagram of Distributed Production, wherein, production station 22 is used to produce urea solution, and it is able to serve and deliver urea solution to the customers 21 around in that service area in a short distance.

The following table 2 indicates the comparisons of characteristics between the Mess production and the Distributed Production.

TABLE 2
comparisons of characteristics between
Mess Production and Distributed Production

| Mess Production | | Distributed Production | |
|---|---|---|---|
| 1. | requiring large area factory 23 to | 1. | production equipment can be put into |
| | perform mess production; | | 20-foot container to move around |
| 2. | the factories 23 are located mostly in | | swiftly; |
| | a remote Industrial Zone; | 2. | requiring less man power to operate; |
| 3. | production process: storing faucet | 3. | providing pre-heating function to |
| | water → reverse osmosis (RO) water | | speed up solubility of urea; |
| | purification → mixing and dissolving → | 4. | production process: storing faucet |
| | resin ions exchange →finished | | water → reverse osmosis (RO) water |
| | product(urea solution); | | purification → pre-heating → mixing |
| 4. | requiring transport vehicle 24 to | | and blending → finished product(urea |
| | make long distance delivery to | | solution); |
| | customer 21, urea solubility time is | 5. | production station can be arranged in |
| | long, cost is high; and | | customer concentrated area to conduct |
| 5. | generating highly concentrated waste | | production and delivery directly; |
| | water in ion exchange process. | 6. | shortening distance to customer 21, to |
| | | | reduce delivery cost; and |
| | | 7. | not requiring ion exchange system, no |
| | | | high concentration waste water |
| | | | generated, the recycled water can be |
| | | | used for ordinary usage. |

The urea solution manufacturing method of the present invention can be used to change the style of urea production in this Industry, since the production station need not occupy excessive space, so it can be put into a container on a container truck for production and move around at fast speed, to overcome the shortcomings of Mess Production in the Prior Art, and to reduce the delivery cost and shorten the delivery time. In particular, the present invention is provided with the water pre-heating capability, to raise urea solubility efficiency by more than 5 times. Presently, in the conventional manufacturing method of the urea solution according to the Prior Art, heating is performed after the urea material is poured into the water. However, according to the physical characteristics of urea material, the Industry grade urea material tends to absorb heat in water, such that once it is poured into water, it will absorb large amount of heat instantaneously to cause temperature of water to drop about 15° C. In other words, in case urea material is poured into water of 45° C., then the water temperature will drop instantaneously to about 30° C. Therefore, in case urea material is poured into water of room temperature of about 20° C. without preheating in advance as done in the Prior Art, then the water temperature may drop to below 10° C., that is quite unfavorable for urea solubility. Since if the temperature of the mixed water and resin is first dropped 15° C., and then it is heated up to over 30° C., that process would take about 2 hours. In the present invention, the water is first pre-heated, then the urea material is poured in, so that the time for its solubility is reduced to 20 minutes.

Summing up the above, the manufacturing method of urea solution used for intelligent and high efficiency diesel engine vehicle of the present invention has the advantages of requiring less production space, easy to move around, high production efficiency, no waste water produced, easy to operate, and low delivery cost, thus it does fulfill patent requirement.

The above detailed description of the preferred embodiment is intended to describe more clearly the characteristics and spirit of the present invention. However, the preferred embodiments disclosed above are not intended to be any restrictions to the scope of the present invention. Conversely, its purpose is to include the various changes and equivalent arrangements which are within the scope of the appended claims.

What is claimed is:

1. A manufacturing method of urea solution used for intelligent and high efficiency diesel engine vehicle, comprising following steps:
step 101: utilizing a reverse osmosis (RO) water purification device to purify faucet water into purified water;
step 102: utilizing a pre-heating device to pre-heat said purified water to 50° C.˜60° C., while keeping that temperature to preheat said purified water;
step 103: adding urea material containing biuret and urea material containing low metal content into said pre-heated water, and using a stirring device to stir them, to make said urea material dissolve into said pre-heated water, to form finished product of pure urea solution used for diesel engine vehicle; and
step 104: storing said finished product of said pure urea solution into a tank, in this approach, through putting said urea material into said pre-heated water to stir and mix them, effect of urea material heat absorption is reduced, to raise urea solubility efficiency 5 times, while reducing production space, such that said production space is moved around swiftly.

2. The manufacturing method of urea solution used for intelligent and high efficiency diesel engine vehicle as claimed in claim 1 wherein said urea solution used for intelligent and high efficiency diesel engine vehicle is put through a filter device to filter out impurities, and then it is stored in a tank.

3. The manufacturing method of urea solution used for intelligent and high efficiency diesel engine vehicle as claimed in claim 1 wherein said pre-heating device is a heat pump heater.
